(19) United States
(12) Patent Application Publication
(10) Pub. No.: US 2001/0034536 A1
(43) Pub. Date: Oct. 25, 2001

(54) Surgical device with malleable shaft

(76) Inventors: Anthony M. Looper, Mount Prospect, IL (US); David Q. Feng, Arlington Heights, IL (US)

Correspondence Address: ALLEGIANCE CORPORATION, ATTN: KIM DILIBERTI, 1430 WAUKEGAN ROAD, MCGAW PARK, IL, 60085-6787, US

(21) Appl. No.: 09/785,374

(22) Filed: Feb. 16, 2001

Related U.S. Application Data
(63) Continuation-in-part of application No. 09/432,523, filed on Nov. 3, 1999.
Application No. 09/432,523 is a continuation of application No. 08/936,394, filed on Sep. 25, 1997, now Pat. No. 6,139,563.

(51) Int. Cl. A61B 17/28

(52) U.S. Cl. 606/205

(57) ABSTRACT

The present invention provides a surgical device having a tissue engaging portion, a shaft member, and a handle assembly. The tissue engaging portion includes first and second opposed jaws for grasping, securing and occluding body tissue and conduits. The shaft member is operatively coupled to the tissue engaging portion and is capable of being placed in different curvatures. The handle assembly is operatively coupled to both the shaft member and to the tissue engaging portion. The shaft member of the present invention allows the surgeon to bend and adjust the shape of the surgical device of minimize its intrusion and to allow for proper positioning in predetermined body locations. In a preferred embodiment, a portion of the device is disposable.

### RELATED CASES

This application is a Continuation-in-part of my co-pending U.S. patent application Ser. No. 9/432,523 filed Nov. 3, 1999, which is a Continuation of U.S. patent application Ser. No. 8/936,394, which is now U.S. Pat. No. 6,139,563.

### FIELD OF THE INVENTION

This invention relates generally to surgical devices and more specifically to a surgical device with a malleable shaft for use in grasping, securing, and occluding body tissue and conduits.

### BACKGROUND OF THE INVENTION

Surgical devices generally include, but are not limited to, clamps, scissors, forceps, dissectors, and retractors. Typically, such surgical devices consist of three elements: a handle, tissue engaging means, and a member extending between the handle and the tissue engaging means. The handle opens and closes the jaws of the tissue engaging means and often has a locking mechanism to hold the jaws closed. The jaws of the tissue engaging means vary extensively in configuration, length, angle, and delicacy depending upon the function of the device and the tissue being engaged. There are many variations of the member provided between the handle and the tissue engaging means. Such members have been provided in a large number of lengths, bends, and angles in order to allow the surgeon to place the jaws in a large number of locations in a wide variety of human body shapes and sizes.

Traditionally, surgeries have been quite invasive to the patient's body, often involving large open incisions. Such surgeries result in great trauma to the patients and require long periods of recovery time. Because these surgeries often involve large incisions, there has not been a strong need for providing surgical devices of a size and detail appropriate for a limited work area. In addition, in order to provide surgeons with a number of choices, surgical devices of various shapes have been provided.

In the recent past, minimally-invasive surgery (MIS) has grown in popularity as an alternative to traditional, large incision surgery. The terms MIS refers to performing surgery in smaller incisions in order to reduce the trauma experienced by the patient, increase the speed of healing, and reduce the recovery time. For the patient, this ultimately equates to less time in the hospital which adds to the cost effectiveness of these procedures.

Understandably, it is very challenging for surgeons to perform surgical tasks in small, MIS incisions. The normal concerns of surgery are compounded with the unique problems brought about by MIS procedures. For example, since the objectives of open surgeries and MIS surgeries are often the same, the occluding of body conduits is still of concern. However, surgical devices of the past were designed for occluding of body conduits during open surgery wherein the size of the surgical device was not constrained by narrow diameters of small, MIS incisions. Thus, such surgical devices, which are necessary in most all procedures, protrude out of the MIS incision and have the potential to interfere with the surgeons' hands as they try to visualize, cut, dissect or suture within the incision. Additionally, in the area of non-minimally invasive surgery, the use of instruments has increased as the surgery techniques have become more and more complex.

Thus, it would be advantageous to have a surgical device which minimizes the degree to which it potentially interferes with the surgeon during any surgery, thereby allowing the surgeon to perform more efficient surgery. It would be further advantageous to have a surgical device that allows proper positioning to predetermined body locations within the small incisions.

### BRIEF DESCRIPTION OF THE INVENTION

The present invention provides a surgical device which minimizes the degree which it potentially interferes with the surgeon during surgery, particularly but not limited to, MIS. The present invention also provides a surgical device that allows proper positioning to predetermined body locations. The present invention achieves these objectives by utilizing a surgical device with a malleable shaft which allows the surgeon to bend and adjust the shape of the device to minimize its intrusion and to allow for proper positioning in predetermined body locations. The surgical device of the present invention is further provided with tissue engaging means and a handle portion.

### BRIEF DESCRIPTION OF THE DRAWINGS

FIG. 1 is a side view of a preferred embodiment made in accordance with the principles of the present invention.

FIG. 2 is a side view of an alternate embodiment of the handle and ratchet assembly of the present invention.

FIG. 2a is a side view of the ratcheting means shown in the assembly of FIG. 2.

FIG. 3 is a side view of an alternate embodiment made in accordance with the principles of the present invention.

FIG. 3a is a side view of the tissue engaging means of the embodiment of FIG. 3, the tissue engaging means being in the closed position.

FIG. 3b is a side view of the tissue engaging means of the embodiment of FIG. 3, the tissue engaging means being in the open position.

FIG. 3c is a cross sectional view of the tissue engaging means of the embodiment of FIG. 3.

FIG. 4 is a cross-sectional view of the mechanism which enables handle to shaft rotation.

FIG. 5 is a cross-sectional view of a malleable embodiment of the shaft member made in accordance with the principles of the present invention.

FIGS. 6a-6c are side views of an alternate embodiment of the jaw actuating mechanism made in accordance with the principles of the present invention.

FIG. 7 is a side view of a wound tubing embodiment of the shaft member made in accordance with the principles of the present invention.

FIG. 7a is a cross-sectional view of the wound tubing embodiment of the shaft member shown in FIG. 7.

FIG. 7b is a cross-sectional view of the wound tubing embodiment of the shaft member shown in FIG. 7, placed in a bent shape.

FIG. 8 is a top view of an alternate embodiment of the jaw actuating mechanism made in accordance with the principles of the present invention.

FIG. 8a is a cross-sectional view of the jaw actuating mechanism shown in FIG. 8, taken along the plane of line 8a-8a.

FIG. 8b is a top view of the jaw actuating mechanism shown in FIG. 8, in the closed position.

FIG. 8c is a top view of the jaw actuating mechanism shown in FIG. 8, shown with alternate jaws.

FIG. 9 is a perspective view of an alternate preferred embodiment made in accordance with the principles of the present invention.

FIG. 9a is a detail view of the ball and socket arrangement used in the embodiment of FIG. 9.

FIG. 10 is a perspective view of a disposable embodiment made in accordance with the principles of the present invention.

FIGS. 11 and 12 are cross-sectional views of the coupling arrangement between the jaw actuating means and the tissue engaging means of the embodiment of FIG. 10.

FIGS. 13 and 14 are cross-sectional views of the coupling arrangement between the jaw actuating means and the handle assembly of the embodiment of FIG. 10.

FIG. 15a is a cross-sectional view of the coupling arrangement of FIGS. 11 and 12, in the locked position.

FIG. 15b is a cross-sectional view taken along the plane of line 15a-15a of FIG. 15a.

FIG. 16a is a cross-sectional view of the coupling arrangement of FIGS. 11 and 12, in the unlocked position.

FIG. 16b is a cross-sectional view taken along the plane of line 16a-16a of FIG. 16a.

FIG. 17 is a cross-sectional view of an alternate embodiment of the jaw actuating mechanism made in accordance with the principles of the present invention.

FIG. 17a is a top view of the jaw actuating mechanism shown in FIG. 17.

FIG. 17b is a cross-sectional view of the jaw actuating mechanism shown in FIG. 17, in the closed position.

FIGS. 18 and 19 are cross-sectional views of an alternate embodiment of the coupling arrangement between the jaw actuating means and the handle assembly made in accordance with the principles of the present invention.

FIG. 18a is an enlarged view of the coupling arrangement of FIG. 18.

FIG. 19a is an enlarged view of the coupling arrangement of FIG. 19.

FIG. 20 is a perspective view of an alternate preferred embodiment of a surgical device made in accordance with the principles of the present invention.

FIG. 21 is a transverse cross-sectional prospective view of a shaft member according to an preferred embodiment of the present invention.

FIG. 22 is a graph of illustrating bending moment versus curvature and bending radius of a tubular shaft member including cross sectional views of the tubular shaft member corresponding to different locations along the graph.

FIG. 23 is a longitudinal cross-sectional view of a shaft member according to an alternative preferred embodiment of the present invention.

FIG. 24 is a transverse cross-sectional view of the shaft member of FIG. 23.

FIG. 25 is a transverse, perspective cross-sectional view of a shaft member according to an alternative preferred embodiment of the present invention.

FIG. 26 is a transverse, perspective cross-sectional view of a shaft member according to an alternative preferred embodiment of the present invention.

FIG. 27 is a perspective view of tissue engaging means of the surgical device of FIG. 20.

FIG. 28 is an exploded view of the tissue engaging means, the shaft member and the jaw actuating means of the surgical device of FIG. 20.

FIG. 29 is an exploded view of the tissue engaging means, the shaft member and the jaw actuating means of the surgical device of FIG. 20.

### DETAILED DESCRIPTION OF THE INVENTION

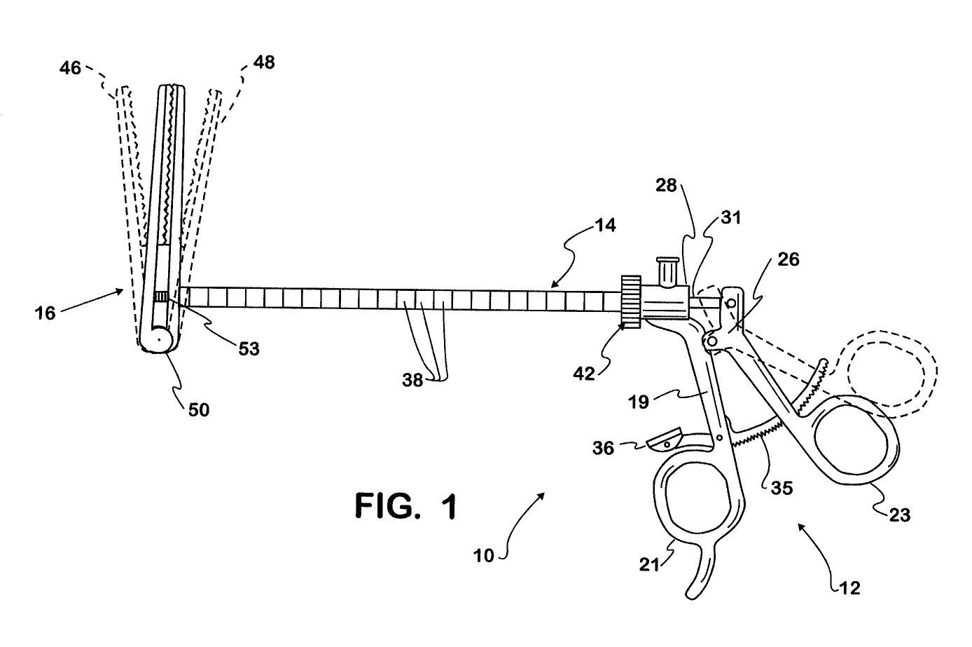

Referring first to FIG. 1, a surgical device 10 made in accordance with the principles of the present invention is shown. The surgical device 10 generally includes a handle portion 12, a shaft member 14, and tissue engaging means 16. Although the figures depict a clamping device, it should be understood that the principles of the present invention are not limited to clamping devices and can be applied to other surgical devices such as, for example, forceps, dissectors, and retractors.

The handle portion 12 functions to move the tissue engaging means 16 between open and closed positions. The handle portion 12 comprises a ratchet handle assembly 19 having an angled handle. It should be understood the alternate handle assemblies having different orientations or ratchet designs could also be employed. The handle assembly 19 of the present invention includes a pair of elongate legs 21, 23 which terminate at distal ends with finger grips and which are pivotably connected together at an intermediate location along the lengths thereof at a pivot element 26. A shaft element 28 for the shaft member 14 is mounted to the proximal end of leg 21. While leg 21 remains stationary with respect to the shaft support element 28, leg 23 moves with respect to leg 21 about the pivot element 26. Additionally, the proximal end of leg 23 is operatively connected to an actuating means 31 which extends axially through the shaft member 14 and is operatively coupled to the tissue engaging means 16. In the preferred embodiment, the actuating means 31 comprises a cable. However, other alternate equivalent actuating means could also be employed.

The handle assembly 19 is further provided with a ratcheting mechanism 35 which is mounted on one of the legs and which interacts with the other leg to hold the tissue engaging means in the closed position. To move the tissue engaging means to the open position, lever 36 is depressed to release leg 23 and the tissue engaging means from the closed position. To move the tissue engaging means from the open position to the closed position, leg 23 is pushed toward leg 21, the proximal end of leg 23 pulling back on the actuating means 31 and thereby actuating the tissue engaging means. Actuating of the tissue engaging means will be discussed in more detail below.

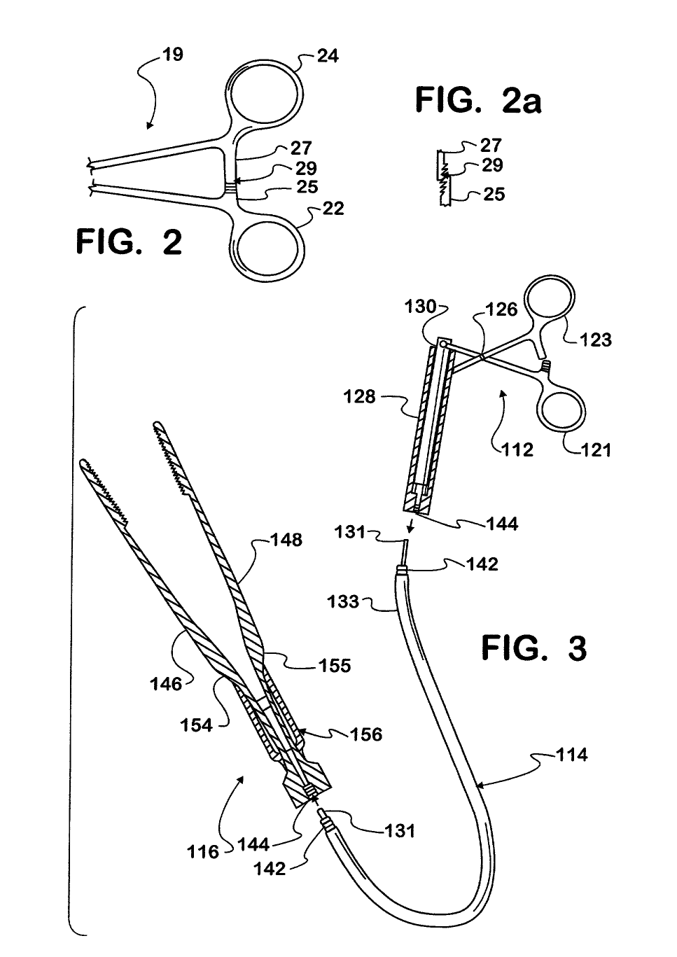

An alternate handle assembly and ratcheting mechanism that could be used with the present invention is shown in FIG. 2. The handle assembly 19 includes two elongate legs 22, 24 operatively coupled together at one end. The legs terminate at distal ends with finger grips. Each of the legs is also provided with a lateral extension 25, 27 carrying ratcheting means 29. The ratcheting means 29 cooperate in the manner shown in FIG. 2a. As the legs are moved relative to one another, the ratcheting means cooperate to set the tissue engaging means of the device in the desired position.

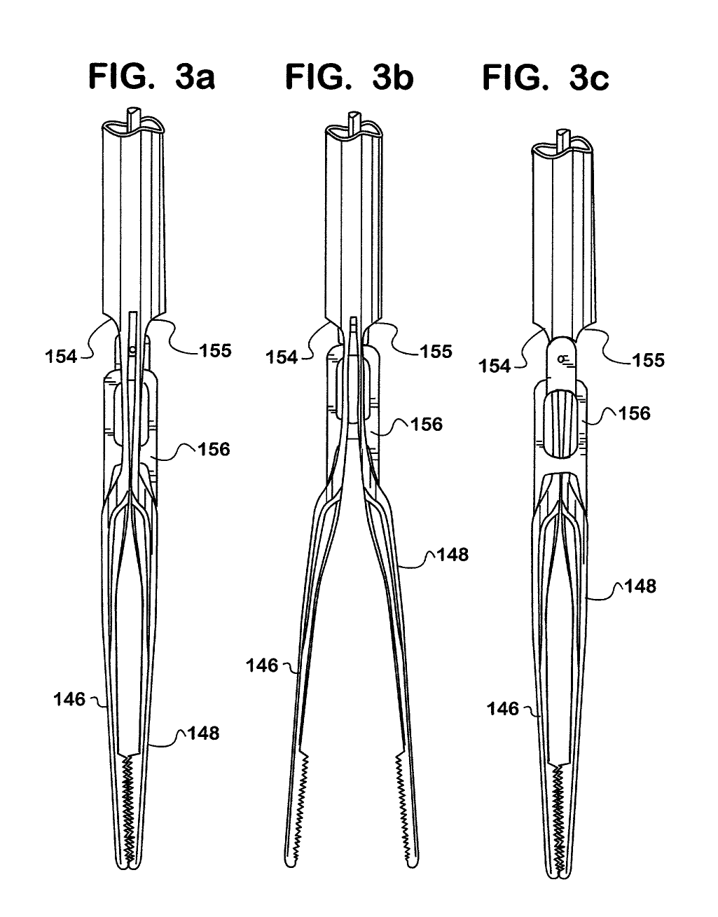

In an alternate embodiment of the handle assembly, shown in FIG. 3, leg 123 can be mounted on a shaft support element 128 for shaft member 114 while leg 121 moves about the pivot element 126. The proximal end of leg 121 is operatively connected to piston 130 which reciprocates axially within shaft support element 128. When leg 121 is moved toward leg 123, leg 121 acts upon piston 130 which in turn pushes on the actuating means 131. The actuating means 131 in turn acts on the tissue engaging means 116.

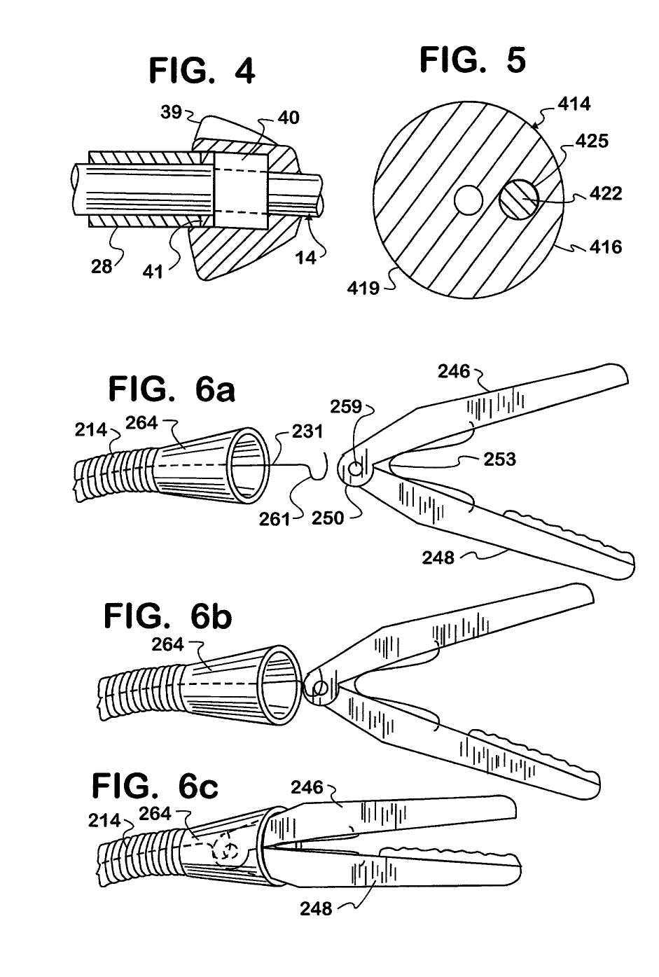

The present invention can also be provided with a mechanism that enables the handle assembly 19 to rotate freely relative to the shaft member 14 to allow the handle to lie flat on the operating table and out of the surgeon's way. FIG. 4 shows the detailed view of this mechanism. As knob 39 is loosened from its attachment with the support element 28 of the handle assembly, the force applied by the knob 39 against bearing 40 and gasket 41 is removed. Consequently, the shaft member 14 can then rotate freely with respect to the support element 28. To set the handle assembly in the desired position with respect to the shaft member, the knob 39 is tightened against the support element 28, thereby acting against the gasket. The gasket 41 thereby functions as a brake, preventing the shaft member to be rotated with respect to the handle assembly after tightening.

The surgical device is further provided with a shaft member 14 which connects the handle assembly 12 to the tissue engaging means 16. As seen in FIG. 1, one end of the shaft member 14 is operatively coupled to the shaft support element 28 of the handle assembly 19 while the opposite end of the shaft member 14 is operatively coupled to the tissues engaging means 16. In the present invention, the shape of the shaft member 14 can be reconfigured in order to enable proper positioning of the tissue engaging means to predetermined body locations. The shaft member 14 can be manipulated to the desired shape to avoid obstructions in an area of work or placed out of the way of the surgeon. It can take a number of forms to accomplish its function.

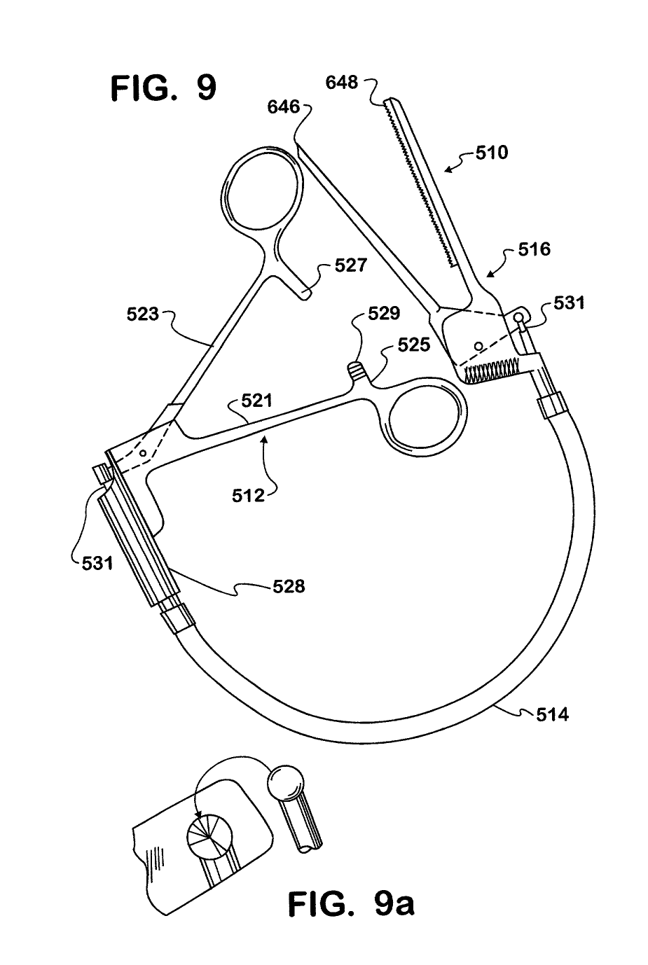

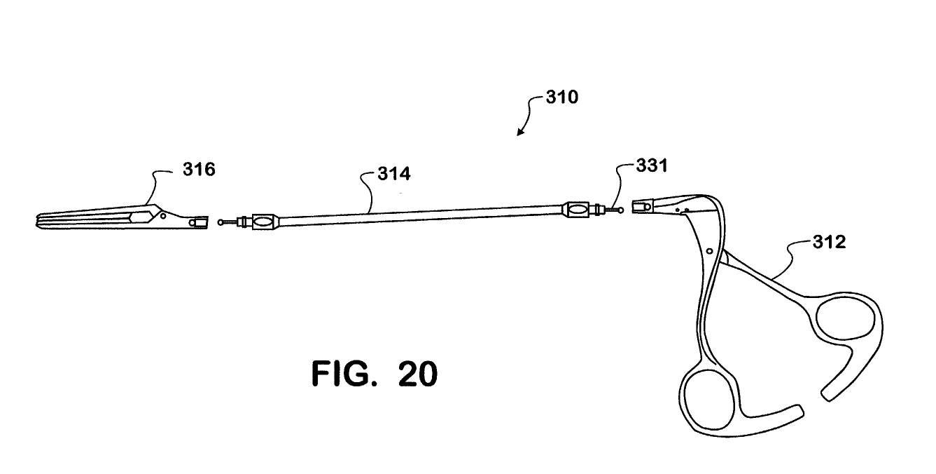

The shaft member can take a malleable form. Due to its malleable nature, the shaft can be placed in various arrangements to reach desired body locations. In such an embodiment of the present invention, shaft member comprises a malleable tube with the actuating means extending axially there through. One end of the actuating means is operatively connected to the tissue engaging means while the other end is operatively coupled to the handle assembly. In one embodiment of the present invention, as shown in FIG. 20, the ends of a jaw actuating means 331 can extend through a shaft member 314 to releasably engage a tissue engaging means 316 and to releasably engage a handle assembly 312 via a ball and socket coupling. Each end of the actuating means 331 is provided with a member in the shape of a ball which mates with a socket carried by the tissue engaging means 316 and the handle assembly 312. FIG. 9a is a detailed view of the ball and socket coupling arrangement between the actuating means and the tissue engaging means. However, alternate equivalent coupling means could also be utilized such as, for example, a cube, a cone, or a disk.

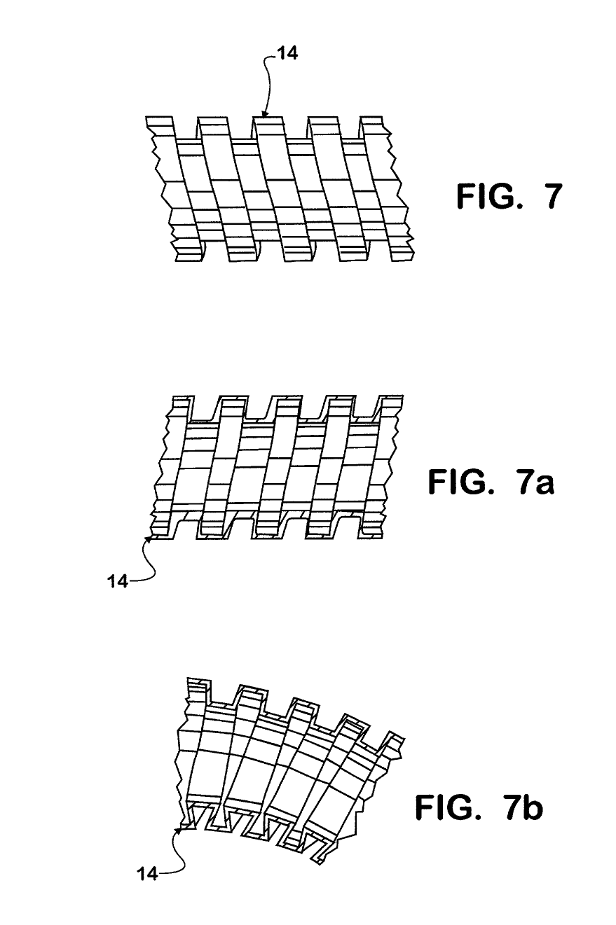

The malleable tube of the shaft member could comprise tubing made of soft metal such as, for example, annealed stainless steel, brass, or aluminum, or wound tubing made of steel that is bendable and that can be placed in different shapes. Such a wound tubing embodiment of the shaft member 14 is depicted in FIGS. 7-7b. For a soft metal tube, the bending moment required to create a permanent set in the shaft in the range of approximately 6 in-lbs to 27 in-lbs, and preferably approximately 10 in-lbs to 15 in-lbs. Alternately, referring to FIG. 5, a shaft member 414 could comprise a dual-channeled tube 416 having the actuating means extending through one channel 419 and a malleable rod 422 extending through the other channel 425 along the length of the tube. The channel 419 housing the jaw actuating means (not shown) preferably extends through the center of the tube 416, with the channel 425 housing the malleable rod 422 extending off-center, as shown in FIG. 5. Alternately, the malleable rod 422 can be positioned in other locations in the tube 416 with respect to its center. Due to the presence of the malleable rod 422, the tube 416 can be placed in various shapes. In a further alternate embodiment, a plurality of malleable rods, rather than a single malleable rod, can be employed to keep the tube in the desired shape.

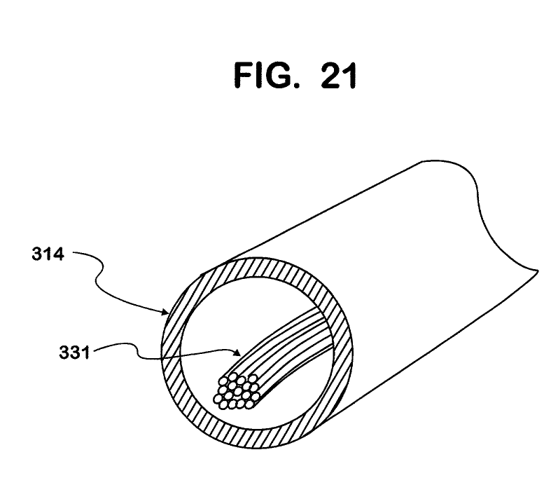

In one preferred embodiment, as shown in FIG. 21, the shaft member 314 is a single walled, elongate tube coupled to the handle assembly 312 (see FIG. 20) at one end and to the tissue engaging means 316 (see FIG. 20) at the other end. The shaft member 314 encloses actuating cable 331, also referred to as a wire driver. The cable 331 extends through the shaft member 314 to prevent moisture and foreign material from contacting the actuating cable 331. The shaft member 314 is made of a malleable material, such as, for example, annealed stainless steel, aluminum, copper, or brass. The shaft member 314 is configured to be positioned into a variety of different shapes, as required by the surgeon, or other user, to avoid obstructions in a work area or to be out of the way of the surgeon.

In an preferred embodiment, the actuating cable 331 is a multi-strand cable. The actuating cable 331 transmits tension and, alternatively, compression from the handle assembly 312 (see FIG. 20) to the tissue engaging means 316. The actuating cable is made of a soft metal 331 such as, for example, stainless steel, aluminum, copper and brass. In an preferred embodiment, the actuating cable 331 is a 19 or a 49 strand stainless steel cable having an outside diameter ranging from about 0.032 inches to about 0.063 inches. Preferably, the outside diameter is approximately 0.047 inches. The cable 331 can be releasable or disposable. Alternatively, the actuating cable 331 can be comprised of a single strand cable or rod.

In an preferred embodiment, the shaft member 314 is sized and configured to be easily bendable, to be highly kink resistant, and to be highly fatigue resistant. The shaft member 314 is easily bendable in response to the application of a reasonable bending moment. A reasonable bending moment is that which an average human can exert to bend the shaft member 314 with minimal effort. The bending moment can range from about 6 to 27 in-lbs and, preferably, is approximately 10-15 in-lbs. The moment (M) produced by the bending of the shaft member 314 can be shown by the following formula:

$$M = \frac{E}{2} π {r}^{3} t [ 2 c - \frac{3 {r}^{4} {c}^{3} ( 1 - {v}^{2} )}{{t}^{2}} ]$$

Where,

E=Young's Modulus

r=the outer radius of the shaft member

t=the wall thickness of the shaft member

v=Poisson's ratio

c=curvature

The curvature (c), is the inverse of the bending radius (R) of the shaft member 514.

c=1/R

The high kink resistance of the shaft member 314 enables the shaft member 314 to be bent and positioned in a variety of different shapes without significantly reducing the inside diameter of the shaft member 314 and, therefore, without causing binding of the actuating cable 531 during operation. The high kink resistance of the shaft member 314 further enables the shaft member 314 to resist kinking above a predetermined minimum bending radius. In an preferred embodiment, the minimum bending radius is about ⅜ of an inch. In an alternative preferred embodiment, the minimum bending radius is about ¼ of an inch. The kink resistance of the shaft member 514 can be shown by the following formula:

$${c}_{kmk} = \frac{\sqrt{2}}{3} \frac{t}{{r}^{2} \sqrt{( 1 - {v}^{2} )}}$$

The ratio of wall thickness (t) to the square of the outer radius (r) of the shaft member 314 (t/r5), is an important segment of the kink resistance formula. A high kink resistance value is desirable. As illustrated by the kink resistance formula, the kink resistance value can be increased through an increase in wall thickness (t) or through a decrease in the outer radius (r) of the shaft member 314. In a preferred embodiment, the kink resistance, and therefore, the thickness (t) and outer radius (r) of the shaft member 314 are sized to produce a maximum moment before kinking. In a preferred embodiment, the moment ranges from approximately 10-15 in-lbs. A preferred range of the ratio of wall thickness (t) to the square of the outer radius (r) of the shaft member 314 (t/r5) of the kink resistance formula is about 2.0 to 6.0. Such a range provides flexibility in material selection for achieving the desired maximum moment of approximately 10-15 in-lbs. If the moment is set too high, the shaft member 314 will be difficult to shape to the desired form. Conversely, a moment set too low may result in buckling of the shaft during a routine “stab wound” procedure. The maximum moment before kinking (Mₘₐₓ) is shown by the following formula:

$${M}_{max} = \frac{2 \sqrt{2}}{9} \frac{E π {rt}^{2}}{\sqrt{( 1 - {v}^{2} )}}$$

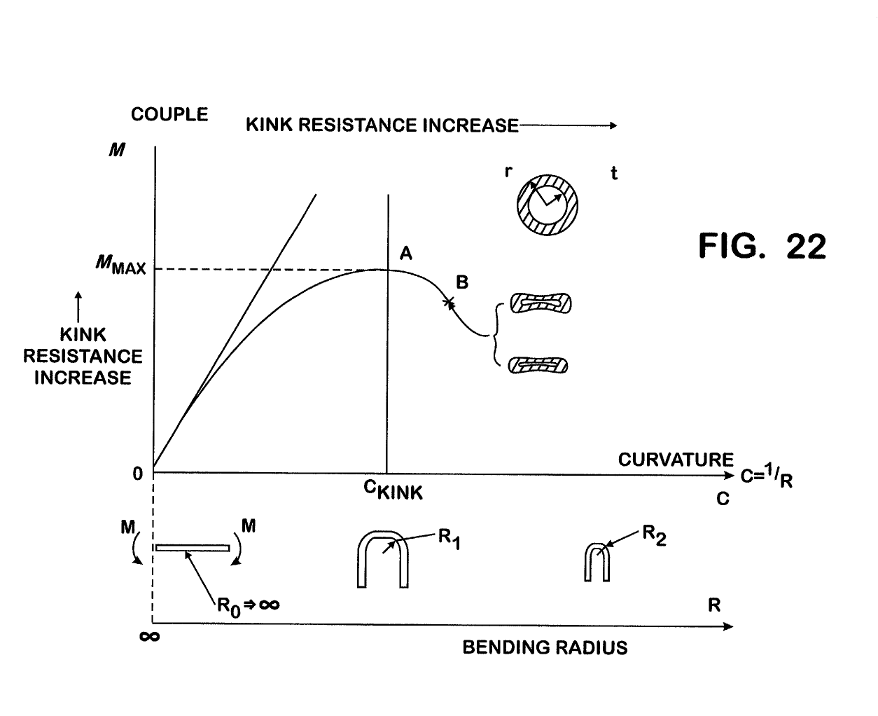

The maximum moment (Mₘₐₓ) identifies the maximum amount of moment applied to a tubular member as a result of bending of the tubular member before the tubular member begins to develop a kink. FIG. 22 is a graphical representation of the curvature and moment applied to a tubular member. FIG. 22 illustrates an initial linear relationship between moment applied to the tubular member and the curvature of the tubular member, followed by a progressively greater increase in curvature for a given increase in the moment applied to the tubular member. Point A of FIG. 22, at Mmax, represents the amount of moment applied to the shaft member and the curvature of the shaft member where kinking initiates. Point B of FIG. 22 represents the value of moment and curvature where the shaft member is substantially kinked, or collapsed. FIG. 22 also includes a second y-axis illustrating the bending radius scale decreasing from an infinite value.

The shaft member 314 preferably has a fatigue resistance that is at least sufficient to withstand multiple bends, even at the same location, along the shaft member 314, during the course of a single procedure. In an preferred embodiment, the shaft member 314 is made of a material having a relatively flat stress/strain curve or a slow work-hardening rate.

In an preferred embodiment, the shaft member 314 is made of annealed stainless steel, such as annealed 304 stainless steel or annealed 305 stainless steel, with an outside diameter ranging from about 0.094 inches to about 0.125 and a wall thickness ranging from about 0.008 inches to about 0.020 inches. In an preferred embodiment, the shaft member 314 is made of fully annealed 304 stainless steel having an outside diameter of approximately 0.109 and an inside diameter of approximately 0.085. Further, alternative dimensions for the inside and outside diameter of the shaft member 314 can be used.

In an alternative preferred embodiment, the shaft member 314 is made of aluminum. Preferably, the shaft member 514, when made of aluminum, has an outside diameter ranging from about 0.125 inches to 0.250 inches, and a wall thickness ranging from about 0.015 inches to 0.050 inches. Because aluminum has a Young's modulus which is over 60% less than stainless steel, aluminum enables the shaft member 314 to include a greater wall thickness while maintaining a similar bending moment. The greater wall thickness improves the kink resistance of the shaft member 314. In alternative preferred embodiments, the shaft member 314 is made of other soft metals, such as copper, brass, etc.

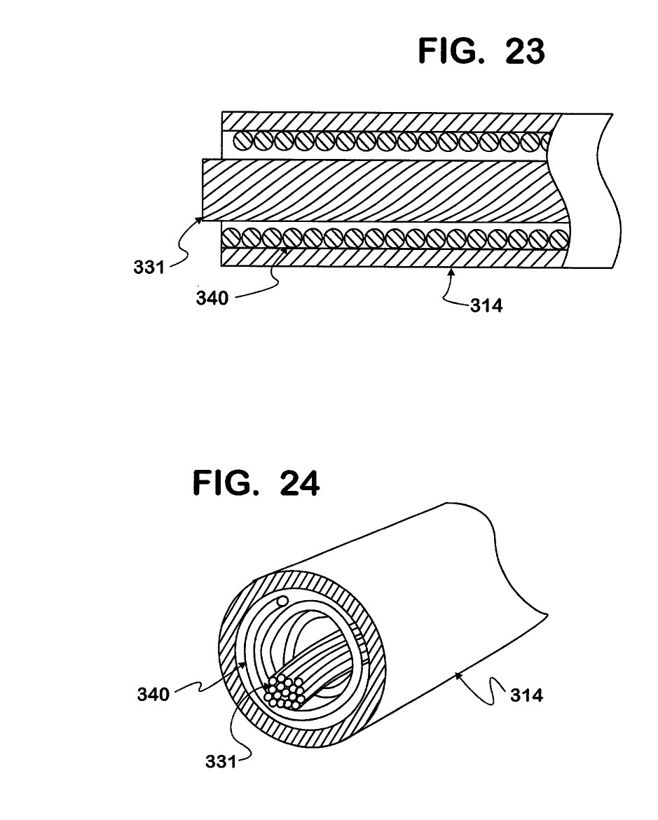

As best shown in FIGS. 23 and 24, in an alternative preferred embodiment, the shaft member 314 includes a spring 340. The spring 340 is a helical spring disposed within and along the shaft member 314. The spring 340 increases the kink resistance and helps to prevent significant collapse of the shaft member 214 during bending. The spring 340 is made of stainless steel. Alternatively, the spring 240 can be made of other materials, such as copper, brass, etc. In an alternative preferred embodiment, a non-helical spring design can be used. In another alternative preferred embodiment, the spring 340 can be comprised of more than one spring with each spring disposed within a separate portion of the shaft member 314. In another alternative preferred embodiment, a plurality of axially aligned annular members can be disposed within and along the inner diameter of the shaft member 314 in lieu of the spring 340.

In a preferred embodiment, the shaft member 314 is made of AL3003-0 aluminum tubing which has a slow work-hardening rate. In alternative preferred embodiments, other soft metals for the shaft member 314 can be used, such as, for example, aluminum, copper, other stainless steels, etc. The shaft member 314, which includes the spring 340, has been shown to be capable of bending at least ten times at 135 degrees around a ¾ inch diameter mandrel without exhibiting any signs of significant fatigue, collapse or kinking.

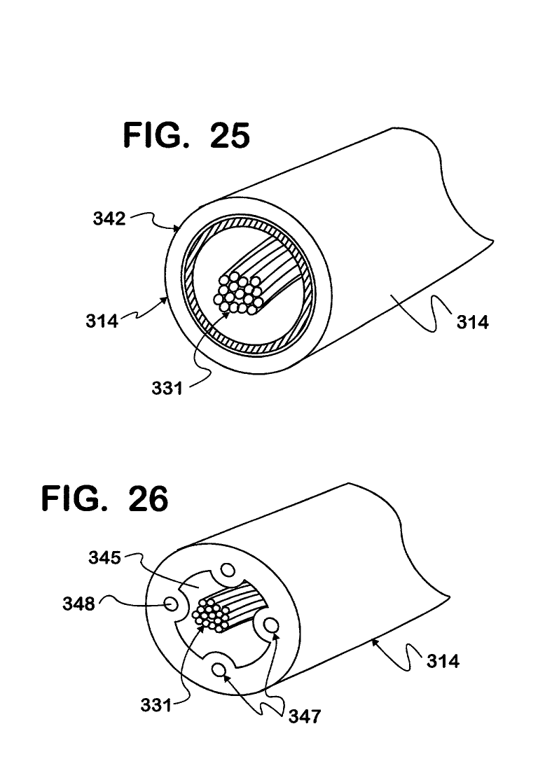

As best shown in FIG. 25, in an alternative preferred embodiment, a second tubular shaft member 342, having an outer diameter which is greater than the outer diameter of the shaft member 314, is coaxially aligned within and along the shaft member 314. The outer shaft (the second tubular shaft member 342) is made of a softer material, having a lower modulus of elasticity, than the material of the inner shaft (shaft member 314). In an preferred embodiment, the outer shaft is made of a soft metal, such as, for example, aluminum, copper, brass, etc., and the inner shaft is made of stainless steel. The inner shaft is configured of harder, stronger material to resist the crushing forces of the outer shaft, as the outer shaft is being bent. The dual shaft configuration of the shaft member 314 and the second tubular shaft member 342 enables the size of the shaft member 314 to be reduced, thereby reducing the bending radius required to cause tube collapse or kinking. The outer shaft adds stiffness to the dual shaft configuration to compensate for the reduced size and the resultant reduced resistance to bending of the shaft member 314. In an alternative preferred embodiment, the shaft member 314 is press fit within the second tubular shaft member 314. In an alternative preferred embodiment, the shaft member 314 and the second tubular shaft member 342 are formed as a co-extrusion, such that no gap is present between the two members. In an alternative preferred embodiment, the outer shaft, (second tubular shaft 342} can be made of other bendable materials, such as, for example, plastic.

In an alternative preferred embodiment, as illustrated in FIG. 26, the shaft member 314 is a multi-lumen plastic tube enclosing the actuating cable 331. The activating cable 331 is operably positioned in an actuating cable lumen 345 and at least one support cable 347 disposed in at least one support lumen 348. The shaft member 314 has a generally cylindrical outer surface and an inner matrix defining the actuating cable lumen 345 and the at least one support lumen 348. Each lumen 345, 348 longitudinally extends within and along the shaft member 314. In an preferred embodiment, the shaft member 314 includes four equally spaced apart support lumens 348, each support lumen 348 including one support cable 347 and the actuating cable lumen 345 is centrally positioned with respect to the support lumens 348. Alternate lumen positions within the shaft member 314 are contemplated. In an preferred embodiment, the shaft member 314 is made of a resilient material, such as, for example, plastic, and the support cables 347 are made of a soft metal, such as, for example, aluminum, brass, copper, nitinol is a nichel-titanium alloy etc. The support cables 347 enable the shaft member 314 to be positionable and malleable.

Alternately, the shaft member comprises a flexible tube with the actuating cable extending axially there through. A second applier instrument that is malleable grasps the shaft member and together the two are inserted into the incision. Once the tissue engaging means are in the closed position, the applier instrument is released and removed.

Referring to FIG. 1, the surgical device is further provided with a tissue engaging means 16 which functions to grasp, secure, and occlude body tissue and conduits. The tissue engaging means 16 includes a pair of jaws 46, 48, the jaws being connected at one end by a hinge 50. The jaws are moveable by various mechanisms between an open position and a closed position. The tissue engaging means can also be provided with a compression return spring 53 to assist the jaws in returning to the open position.

Since it is important to surgeons to reduce the size and bulk of the hinge of the jaws in order to increase visualization and to minimize the space the tissue engaging means occupies, in one embodiment of the present invention, the jaws are coaxial with the longitudinal axis of the shaft member. This orientation, which is shown in FIGS. 3, 6, 8 and 9, reduces the size and bulk of the hinge while still maintaining the strength required by the jaws. However, the tissue engaging means can be placed in alternate arrangements with respect to the shaft member. For example, in FIG. 1, the tissue engaging means is arranged at approximately a 90° angle with respect to the shaft member.

In one embodiment of the invention, the shaft member is separable from the tissue engaging means. In use, the shaft member is utilized to place the tissue engaging means in the location desired. The shaft member is then released from the tissue engaging means and removed from the patient's body, leaving the tissue engaging means within the body. The tissue engaging means has a suture or tether attached to it, which extends out of the incision. When the tissue engaging means is to be removed, the shaft member is inserted back into the incision and is guided to the tissue engaging means by the suture or tether. The shaft member then is coupled to the tissue engaging means and the entire device is removed.

The jaws can be actuated by a number of different mechanisms, as shown in FIGS. 3, 6, 8, 9, and 10. Despite the use of a non-rigid shaft member, the present invention is capable of exerting a force on the tissue engaging means in the range of approximately 10-20 lbs. In the embodiment of FIG. 3, the hinged end of each jaw is provided with a reduce thickness portion 154, 155. In the open position of the tissue engaging means, shown in FIG. 3b, a jaw actuating member 156 mates with the reduced thickness portions of the jaws. In use, the handle assembly 112 is actuated, thereby pushing the actuating means 131 forward. The actuating means 131 in turn pushes the actuating member 156, thereby causing it to slide forward and out of the reduced thickness portions, as shown in FIG. 3a. This motion squeezes the jaws 146, 148 to the closed position while the reverse motion separates the jaws 146, 148 to the open position.

In the alternate embodiment of FIGS. 6a to 6c, the hinged end of each jaw is provided with a hole 259 which interacts with a hook 261 provided at the end of the actuating cable 231. Spring 253 is provided to maintain the jaws 246, 248 in the open position, as shown in FIG. 6a. To place the jaws 246, 248 in the closed position, the handle assembly is actuated, thereby pulling the cable 231 and hook 261 back through the shaft member 214. As the cable 231 is pulled back, the jaws 246, 248 are actuated to the closed position by their interaction with a conical end member 264 provided on the shaft member 214.

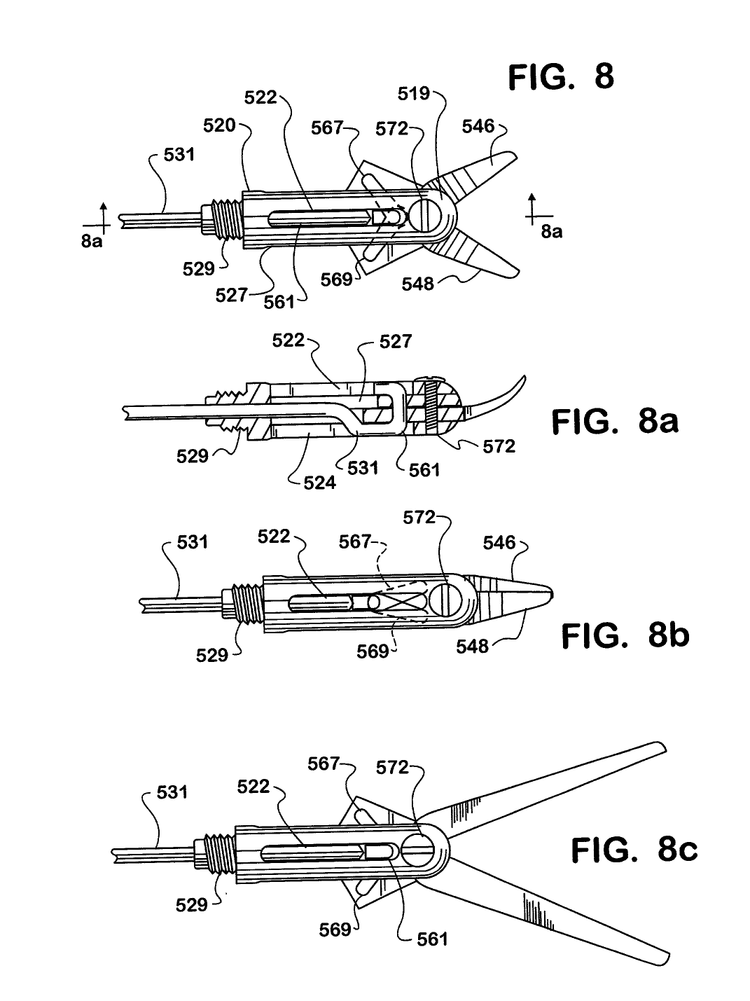

FIG. 8 depicts a further alternate embodiment of the jaw actuating mechanism. The mechanism includes a cylindrical clevis 520 having two longitudinal slots 522, 524 along its length, the slots located opposite of one another. The clevis 520 further includes a longitudinal cut-out 527 along its length. The jaws 546, 548 are disposed at one end of the clevis 520. The opposite end of the clevis 520 is provided with a cylindrical extension 529 through which the jaw actuating mechanism extends. In this embodiment, the jaw actuating mechanism comprises the wire driver 531 which extends through the cylindrical extension 529 and is operatively connected to one end of the jaws.

The jaws 546, 548 of this embodiment are provided with a diagonal slot 567, 569 at one end. As shown in FIG. 8b, the slotted ends of the jaws are disposed within the cut-out 527 of the clevis when the jaws are in the closed position. The jaws are attached along their median portion to the clevis by a screw 572 extending transversely across the longitudinal cut-out 527. The remainder of the jaws, the tissue engaging ends, extend from the clevis 520.

The end of the wire driver 531 which is coupled to the jaws 546, 548 is provided with a hook 561. As seen in FIG. 8a, a portion of the hook 561 is accommodated within each of the longitudinal slots. The remaining portion of the hook is coupled to the slots 567, 569 of the jaws. To actuate the jaws to an open position, the driver 531 is pushed toward the jaws. This motion causes the hook 561 to travel to one end of each of the slots 567, 569, thereby causing the jaws 546, 548 to pivot about the screw and move to the open position. As shown in FIG. 8, the slotted ends of the jaws extended outwardly from the longitudinal cut-out 527 when the jaws are in the open position. To return the jaws back to the closed position, the driver 531 is moved in the direction away from the jaws, thereby causing the driver to move to the opposite end of the slots 567, 569. The jaws again move about the screw to the closed position. In the closed position, the slotted ends of the jaws are within the cut-out 527. As can be seen by referring to FIGS. 8 and 8c, this type of actuating mechanism can be used with different tissue engaging means.

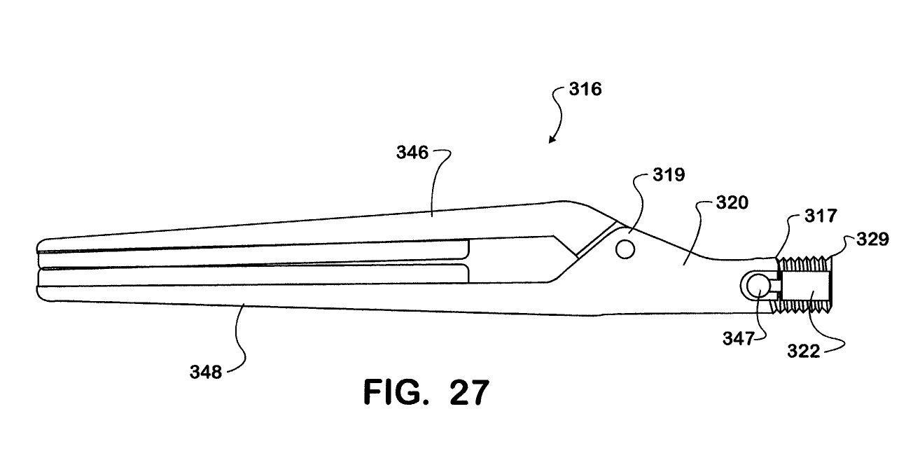

FIGS. 20 and 27 through 29 depict a further alternate embodiment of the surgical device indicated as 310 including the handle assembly 312, the shaft member 314 and tissue engaging means 36. Referring to FIG. 27, tissue engaging means 316 includes a cylindrical clevis 320 having a proximal end 317 and a distal end 319. First and second jaws 346, 348 are disposed at the distal end 319 of the clevis 320. The proximal end 317 of the clevis 320 is provided with a cylindrical extension 329 through which the jaw actuating mechanism 331 extends. The jaw actuating mechanism 331 extends through the cylindrical extension 329 and is operatively connected to the first jaw 346. The clevis 320 includes a slot 322 longitudinally extending from the proximal end 317 toward the jaws 346, 348. The first jaw 346 preferably includes a proximal end having socket 347. The socket 347 is configured to releasably engage a distal end of the jaw actuating means 331.

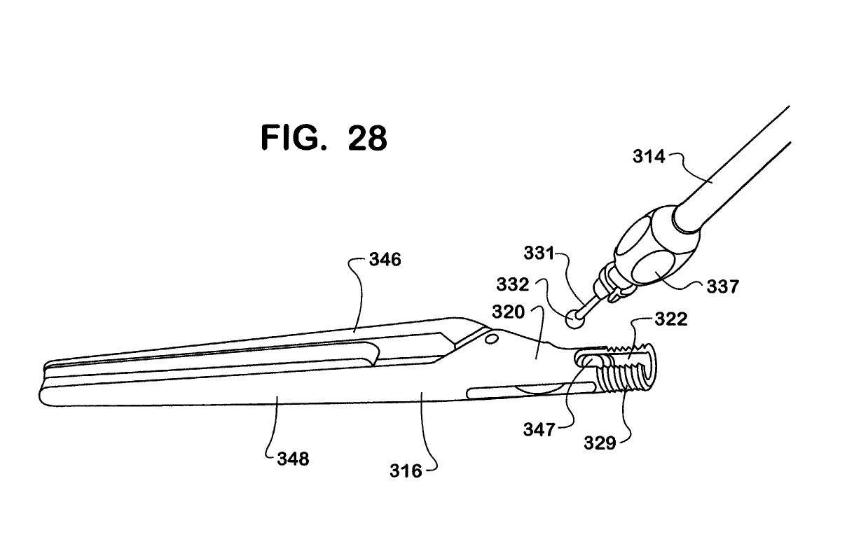

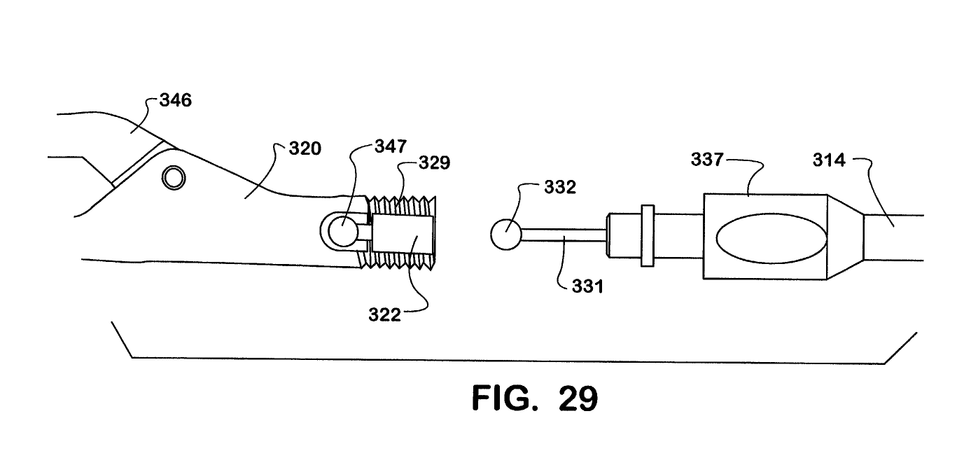

Referring to FIGS. 28 and 29, the jaw actuating means 331 includes a ball 332 connected to the distal end of the jaw actuating means 331. The ball 332 and the distal end of the jaw actuating means 331 are configured to extend through the slot 322 of the clevis 320 and the ball is configured to releasably engage the socket 347 of the first jaw 346. The jaw actuating means 331 further includes a cap 337 for connecting to the cylindrical extension 329 of the clevis 320. Preferably, the cylindrical extension 329 of the clevis 320 and the cap 337 are threadedly engaged. Alternative shapes for the ball 332 of the jaw actuating means 331 and its mating socket 347 are contemplated such as, for example, a cube, a cone or a disk.

A further preferred alternate embodiment of the present invention is depicted in FIG. 9. The surgical device 510 generally includes a handle assembly 512 comprising shaft support 528 and legs 521, 523, a shaft member 514 with an actuating cable 531 extending therethrough, and tissue engaging means 516 including jaws 646, 648. The actuating cable 531 is coupled to the leg 523 and to jaw 646 by a ball and socket arrangement, as mentioned above and as shown in detail in FIG. 9a. When leg 523 of the handle assembly is moved toward leg 521, this movement pulls on the actuating cable 531. The actuating cable 531 in turn pulls jaw 646, causing it to move toward jaw 648 and to the closed position. When it is desired to return the jaws to the opened position, leg 523 is released, thereby releasing the force on the cable and returning the jaws to the open position.

In the embodiment of FIG. 9, the shaft member 514 can take one of two forms. The shaft member 514 can comprise the dual-channeled tube, as discussed above and shown in FIG. 5. Alternately, the shaft member can be comprised of tubing made of soft metal such as, for example, annealed stainless steel, brass or aluminum that is bendable and that can be placed in different shapes. In either instance, the shaft member is of a malleable type so that it can be placed in various arrangements to reach desired body locations.

In order to save the time and costs involved in sterilizing the surgical device and to reduce the cost and waste involved with fully disposable devices, the surgical device of the present invention can be made in part of disposable material so that the remainder of the surgical device is reusable. In one embodiment of the present invention, the actuating cable and the shaft member are made of disposable material, and handle portion and the tissue engaging means are made of re-useable material such as stainless steel.

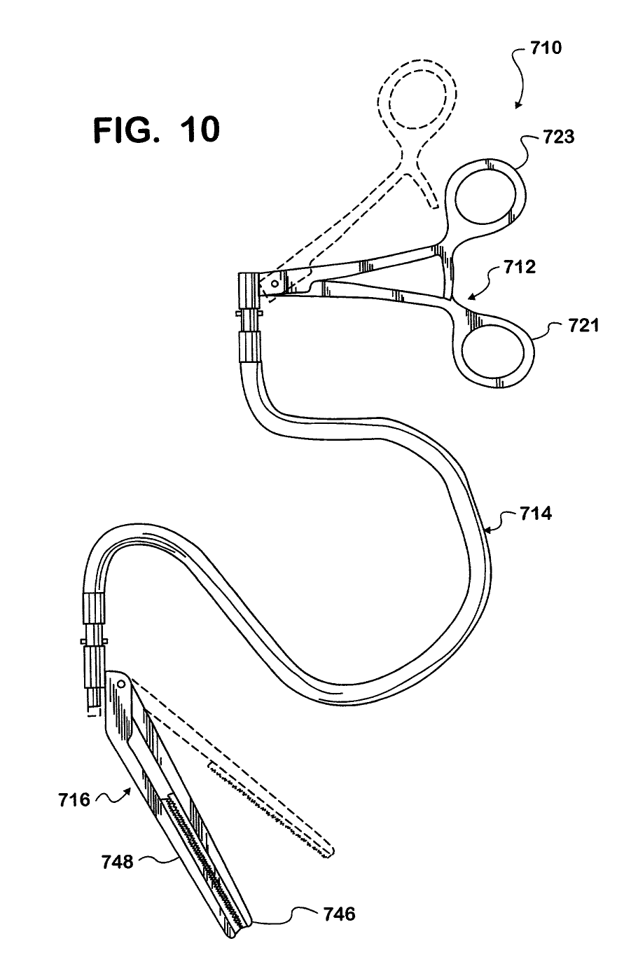

One such device is shown in FIG. 10. The device 710 includes a disposable shaft member 714 operatively coupled to the tissue engaging means 716 and to the handle assembly 712, both made of re-useable material. The shaft member 714 is comprised of a malleable tube, preferably of a soft metal such as, for example, annealed stainless steel, brass or aluminum, having a plastic covering or a powder/paint coating for cosmetic purposes. Alternatively, the shaft member 714 is comprised of a malleable plastic tube, preferably of polyethylene or some other suitable plastic extrusion. In additional alternative embodiments, any combination of one or more of the shaft members, the actuating cable, the tissue engaging means and the handle assembly can be disposable and the remaining components can be reusable.

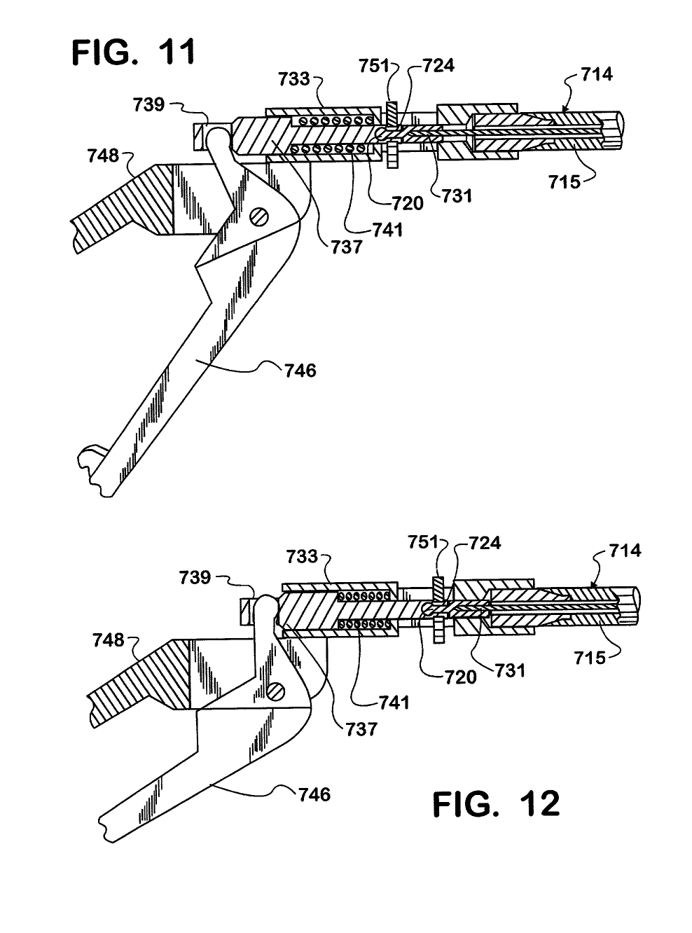

Referring to FIG. 11, a jaw actuating means 731, comprising either a flexible cable or rod, extends through the tube 715, the actuating means 731 being capable of sliding freely within the tube. Each end of the actuating means 731 extends from a respective end of the malleable tube 715 and is preferably provided with a spherical ball 720, 722 at its tip. As shown in FIGS. 11-14, both the tissue engaging means 716 and the handle assembly 712 are provided with a mating socket 724, 726 for the spherical ball 720, 722. As discussed below, the malleable tube 715 is coupled to the tissue engaging means 716 and to the handle assembly 712 by the mating of the spherical balls 720, 722 with the sockets 724, 726. Alternative shapes for the tip of the malleable tube and its mating socket tip are contemplated such as, for example, a cube, a cone or a disk.

Referring to FIGS. 11 and 12, one of the jaws 748 of the tissue engaging means 716 is provided at one end with a cylinder 733. The other jaw 746 of the tissue engaging means 716 is provided with a bolt 737. The bolt 737 includes a cut-out portion 739 in which one end of jaw 746 pivots. The bolt 737 then extends away from jaw 746 through the cylinder 733 to mate with the jaw actuating means 731. As mentioned above, the bolt 737 is provided with the socket 724 which mates with the spherical ball 720 of the jaw actuating means 731. The cylinder 733 is also provided with a spring 741 which biases the bolt 737 and in turn the jaw 746 to the open position. To actuate the jaws of the tissue engaging means from the open position shown in FIG. 11 to a closed position, the handle assembly 712 is actuated to pull one end of the jaw actuating means 731 in the direction away from the tissue engaging means 716. Due to the ball and socket coupling, the bolt 737 is also pulled away from the tissue engaging means 716. This action causes the bolt 737 to act on jaw 746 via the cut-out portion 739, the jaw 746 pivoting to the closed position shown in FIG. 12. Since the jaws are spring biased to the open position, upon release of the pressure on the legs of the handle assembly, the jaws are returned to the open position.

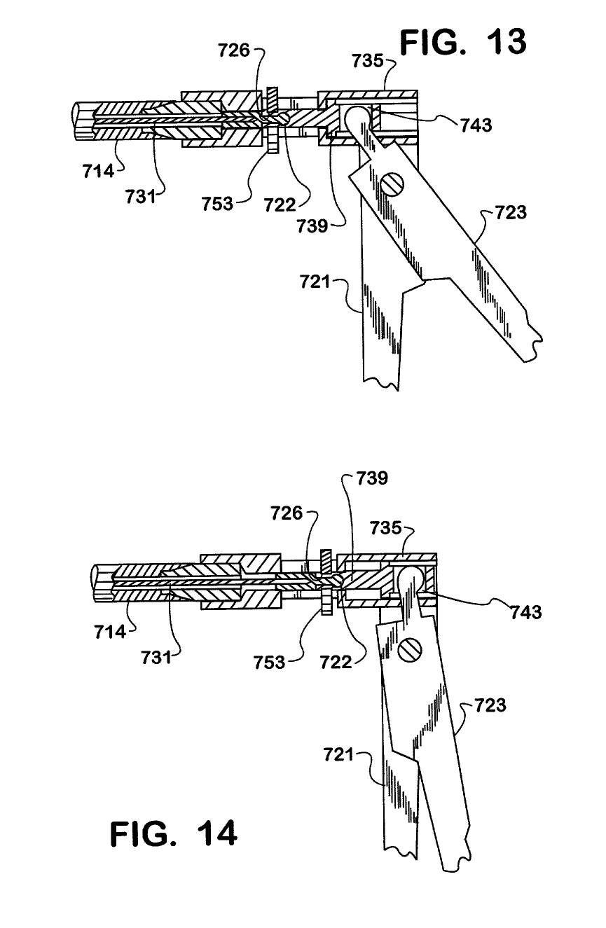

As shown in FIGS. 13 and 14, a similar ball and socket coupling is employed to couple the jaw actuating means 731 to the handle assembly 712. One of the legs of the handle assembly 712 is provided at one end with a cylinder 735 while the other leg 723 is provided with a bolt 739. The bolt 739 includes a cut-out portion 743 in which the end of leg 723 pivots. The bolt 739 then extends away from the leg 723 through the cylinder 735 to mate with the jaw actuating means 731. To mate with the spherical ball 722 of the jaw means 731, the bolt 739 is provided with a socket 726. In order to actuate the tissue engaging means 716 to the closed position, the legs 721, 723 of the handle assembly 712 are moved from the position shown in FIG. 13, in which the legs are apart from one another, to the position shown in FIG. 14, in which the legs are brought together. With this action, leg 723 acts on the bolt 739 pulling it in a direction away from the tissue engaging means 716. The bolt 739 in turn acts on the jaw actuating means 731, pulling it in a direction away from the tissue engaging means 716. As discussed above, this action causes the opposite end of the jaw actuating means 731 to act on jaw 746 of the tissue engaging means 716, thereby bringing the jaws together to the closed position, as shown in FIG. 12.

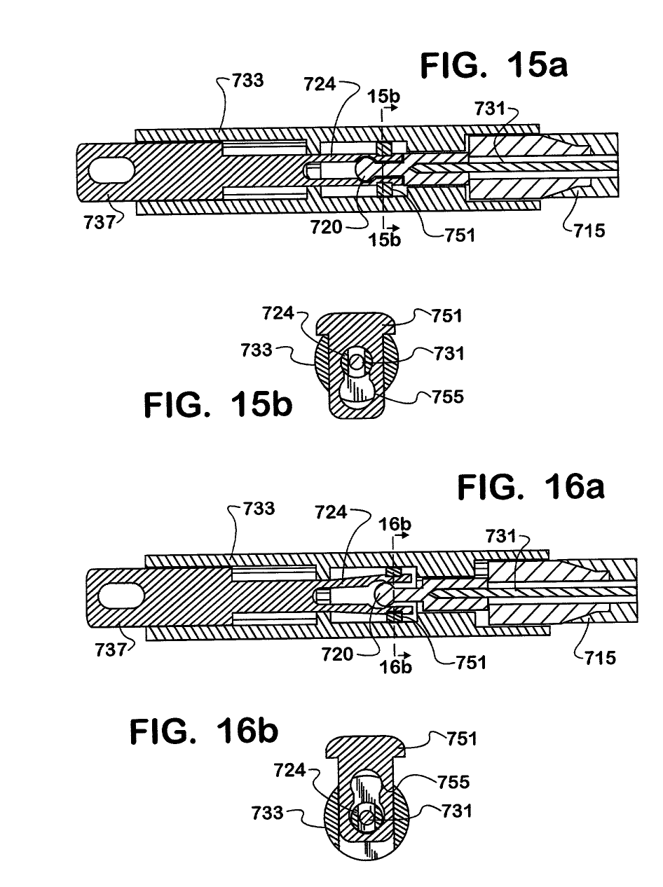

To enable the shaft member 714 to be separated from the handle assembly 712 and the tissue engaging means 716 and thus disposable, locking clips 751, 753 are provided at each ball and socket coupling. As seen from FIGS. 15b and 16b, each clip is provided with an opening 755 generally in the shape of the numeral “8.” To lock the ball 720 of the jaw actuating means 731 to the socket 724 of the bolt 737, the locking clip 751 is placed in the position shown in FIG. 15b. To unlock the ball 720 of the jaw actuating means 731 from the socket 724 of the bolt 737 and thus allow the shaft member 714 to be separated and disposed, the locking clip 751 is placed in the position shown in FIG. 16b.

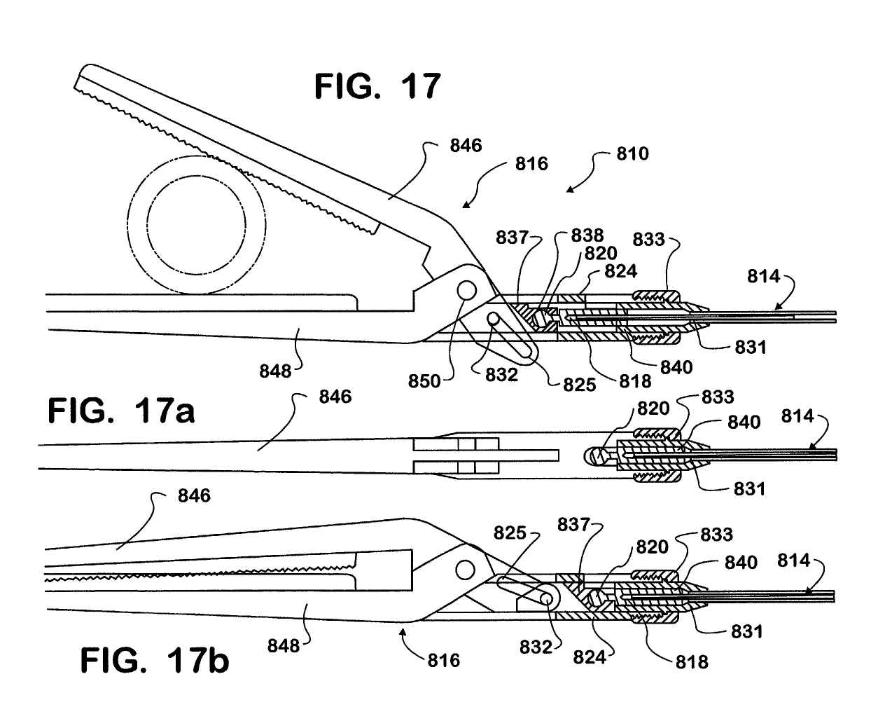

Another preferred alternate embodiment of the present invention is depicted in FIGS. 17-19. The device 810 includes a tissue engaging means 816 with jaws 846, 848, a shaft member 814, and a handle assembly 812 with handles 821, 823.

The shaft member 814 is comprised of a malleable tube, preferably of a soft metal such as, for example, annealed stainless steel, brass or aluminum. Each end of the shaft member is provided with a terminal member 840, 841. As shown in the figures, a jaw actuating means 81 comprising a cable extends through the shaft member 814. The jaw actuating means is provided at each end with a terminal member 818, 819, each terminal member having a spherical ball 820, 822 associated therewith.

Referring to FIGS. 17-17b, the tissue engaging means 816 is carried by a housing 824. Within the housing 824, a bolt member 834 moves, the bolt member being provided with a pin 832 and a socket 838 which cooperates with the spherical ball 820 of the jaw actuating means 831. Jaw 846 of the tissue engaging means is provided with a slot 825 which is operatively coupled to a bolt member 834 via pin 832. The shaft member 814 is coupled to the housing 824 by the cooperation between terminal member 840 and cap 833. To couple the shaft member with the housing, terminal member 840 is abutted against the housing 840 and the cap 833 is then attached to housing 824 via a suitable means such as screw threads.

To actuate the tissue engaging means from its open position, shown in FIG. 17, to its closed position, shown in FIG. 17b, the jaw actuating means is pulled in a direction away from the tissue engaging means. This movement in turn causes the bolt member 837 to move away from the tissue engaging means. As the bolt member moves away, the pin 832 travels from one end of the slot to the other, thereby causing the jaw to pivot about the fulcrum 850 to its closed position. The coupling arrangement between the jaw actuating means and the tissue engaging means allows the force required to remain relatively low, particularly when taking into consideration the long, thin configuration of the shaft member. Once the jaw actuating means is released, it moves back towards the jaw actuating means and the jaw 846 returns to its open position.

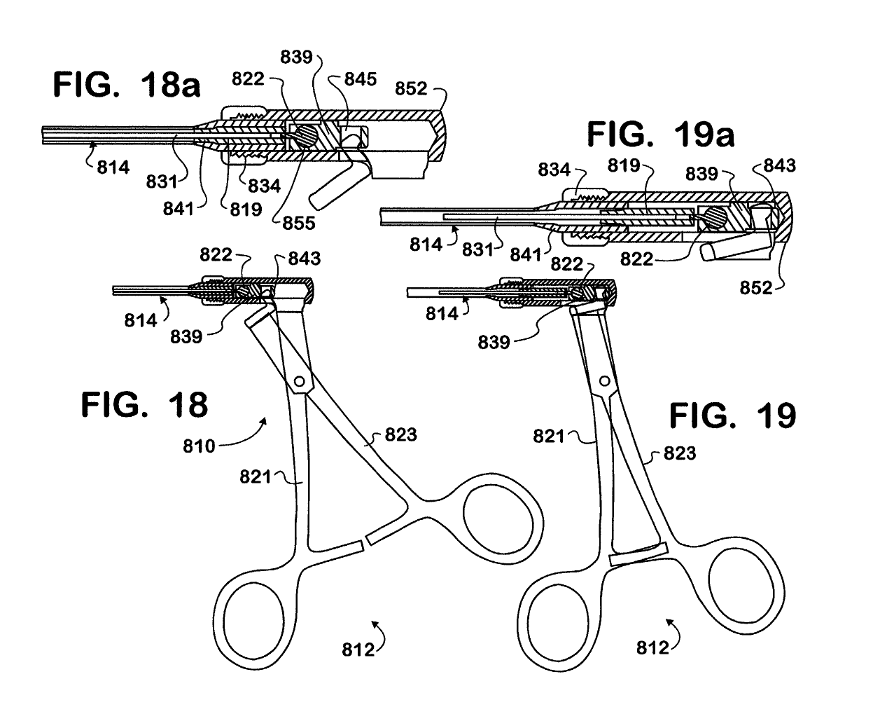

Referring to FIGS. 18-19, handle 821 of the handle assembly is provided at one end with a housing 852. Within the housing 852, a bolt member 839 moves, the bolt member 839 being provided with a socket 855 for coupling with the jaw actuating means and a cut-out portion 843 in which the end of leg 823 pivots. The shaft member 814 is coupled to the housing 852 by the cooperation between terminal member 841 and cap 834. To couple the shaft member 814 with the housing 852, terminal member 841 is abutted against the housing 852 and the cap 834 is then attached to housing 852 via a suitable means such as screw threads.

In order to actuate the tissue engaging means 816 to the closed position, the legs 821, 823 of the handle assembly 812 are moved from the position shown in FIG. 18, in which the legs are apart from one another, to the position shown in FIG. 19, in which the legs are brought together. With this action, leg 823 acts on the bolt member 839 pulling it in a direction away from the tissue engaging means 816. The bolt member 839 in turn acts on the jaw actuating means 831, pulling it in a direction away from the tissue engaging means 816. As discussed above, this action causes the opposite end of the jaw actuating means 831 to act on jaw 846 of the tissue engaging means 816, thereby bringing the jaws together to the close position, as shown in FIG. 17b.

It should be understood that various changes in modifications to the preferred embodiment described herein will be apparent to those skilled in the art. Such changes and modifications can be made without departing from the spirit and scope of the present invention and without diminishing its attendant advantages. It is therefore intended that such changes and modifications be within the scope of the claims.

What is claimed is:

1. A malleable shaft member for a surgical device having a tissue engaging means and a handle assembly, and an actuating means connecting the handle assembly and the tissue engaging means for actuating the tissue engaging means, the shaft member comprising:
a first tube made of a malleable material and having a proximal end, a distal end and a longitudinal axis, the proximal end of the first tube coupled to the handle assembly, the distal end of the first tube coupled to the tissue engaging means, the actuating means extending axially through the first tube, the first tube configured to be kink resistant, fatigue resistant, and to bend about some bending radius in response to a bending moment applied to the first tube.

2. The shaft member of claim 1 wherein the bending moment applied to the first tube ranges between about 6 in-lbs to 27 in-lbs.

3. The shaft member of claim 1 wherein the bending moment applied to the first tube ranges between about 12 in-lbs to 15 in-lbs.

4. The shaft member of claim 1, wherein the minimum bending radius of the first tube ranges from about ¼ inch to ⅜ inch.

5. The shaft member of claim 1, wherein the first tube has a wall thickness and an outer radius extending from the longitudinal axis of the first tube to an outer surface of the first tube, and wherein a ratio of the wall thickness to the square of the outer radius approximately ranges between about 2.0 and about 6.0.

6. The shaft member of claim 1, wherein the first tube is made of a material selected from the group consisting of stainless steel, copper, aluminum and brass.

7. The shaft member of claim 1, wherein the tube has a wall thickness ranging approximately between 0.008 inches and 0.050 inches and an outside diameter ranging approximately between 0.094 inches to 0.125 inches.

8. The shaft member of claim 1, wherein the proximal end of the first tube is removably coupled to the handle assembly.

9. The shaft member of claim 1, wherein the distal end of the first tube is removably coupled to the tissue engaging means.

10. The shaft member of claim 1, further comprising a second tube, the first tube coaxially aligned and disposed within the second tube.

11. The shaft member of claim 10, wherein the second tube is made of a material selected from the group consisting of aluminum, brass, copper and plastic.

12. The shaft member of claim 10, wherein the first tube and the second tube are formed as a co-extrusion.

13. A surgical device comprising:
a tissue engaging means;
a handle assembly;
an actuating means connecting the handle assembly and the tissue engaging means for actuating the tissue engaging means; and
a shaft member made of a malleable material and having a proximal end, a distal end and a longitudinal axis, the proximal end of the shaft member coupled to the handle assembly, the distal end of the shaft member coupled to the tissue engaging means, the actuating means extending axially through the shaft member, the shaft member configured to be kink resistant, fatigue resistant, and to bend about some bending radius in response to a bending moment applied to the shaft member.

14. The surgical device of claim 13, wherein the bending moment applied to the shaft member ranges between 6 in-lbs to 27 in-lbs.

15. The surgical device of claim 13, wherein the bending moment applied to the shaft member ranges between 14 in-lbs to 15 in-lbs.

16. The surgical device of claim 13, wherein the shaft member has a wall thickness and an outer radius extending from the longitudinal axis of the shaft member to an outer surface of the shaft member, and wherein a ratio of the wall thickness to the square of the outer radius approximately ranges between 2.0 and 6.0.

17. The surgical device of claim 13, wherein the shaft member is made of a material selected from the group consisting of stainless steel, copper, aluminum and brass.

18. The surgical device of claim 13, wherein the proximal end of the shaft member is removably coupled to the handle assembly.

19. The surgical device of claim 13, wherein the distal end of the shaft member is removably coupled to the tissue engaging means.

20. The surgical device of claim 13, further comprising an outer tube, the shaft member coaxially aligned and disposed within the outer tube.

21. The surgical device of claim 20, wherein the outer tube is made of a material selected from the group consisting of aluminum, brass, copper and plastic.

22. A malleable shaft member for a surgical device having a tissue engaging means and a handle assembly, and an actuating means connecting the handle assembly and the tissue engaging means for actuating the tissue engaging means, the shaft member comprising:
a tube made of a malleable material and having a proximal end, a distal end and a longitudinal axis, the proximal end of the tube coupled to the handle assembly, the distal end of the tube coupled to the tissue engaging means, the tube configured to be kink resistant, fatigue resistant; and
at least one spring disposed within the tube, the actuating means extending axially through the spring and the tube for inhibiting the collapse of the tube.

23. The shaft member of claim 22, wherein the spring is a helical spring.

24. The shaft member of claim 22, wherein the at least one spring is a single helical spring extending from the proximal end of the tube to the distal end of the tube.

25. The shaft member of claim 22, wherein at least two springs are axially aligned and extend from the proximal end of the tube to the distal end of the tube.

26. The shaft member of claim 22, wherein the tube is made of a material selected from the group consisting of stainless steel, copper, aluminum and brass.

27. The shaft member of claim 22, wherein the proximal end of the tube is removably coupled to the handle assembly.

28. The shaft member of claim 22, wherein the distal end of the tube is removably coupled to the tissue engaging means.

29. A surgical device comprising:
a tissue engaging means;
a handle assembly;
an actuating means connecting the handle assembly and the tissue engaging means for actuating the tissue engaging means; and
a shaft member including:
a tube made of a malleable material and having a proximal end, a distal end and a longitudinal axis, the proximal end of the tube coupled to the handle assembly, the distal end of the tube coupled to the tissue engaging means, the tube configured to be kink resistant, fatigue resistant; and
at least one spring disposed within the tube, the actuating means extending axially through the spring and the tube.

30. The surgical device of claim 29, wherein the spring is a helical spring.

31. The surgical device of claim 29, wherein the at least one spring is a single helical spring extending from the proximal end of the tube to the distal end of the tube.

32. The surgical device of claim 29, wherein the tube is made of a material selected from the group consisting of stainless steel, copper, aluminum and brass.

33. The surgical device of claim 29, wherein the proximal end of the tube is removably coupled to the handle assembly.

34. The surgical device of claim 29, wherein the distal end of the tube is removably coupled to the tissue engaging means.

35. A malleable shaft member for a surgical device having a tissue engaging means and a handle assembly, and an actuating means connecting the handle assembly and the tissue engaging means for actuating the tissue engaging means, the shaft member comprising:
an outer tube having a proximal end, a distal end and a longitudinal axis; and
a inner tube made of a malleable material and having a proximal end, a distal end and a longitudinal axis, the proximal end of the inner tube coupled to the handle assembly, the distal end of the inner tube coupled to the tissue engaging means, the actuating means extending axially through the inner tube, the inner tube coaxially aligned and disposed within the outer tube, the inner tube configured to be kink resistant, fatigue resistant, and to bend about a bending radius in response to a bending moment applied to the inner tube.

36. The shaft member of claim 35, wherein the outer tube is made of a material selected from the group consisting of aluminum, brass, copper and plastic, and wherein the inner tube is made of a material selected from the group consisting of stainless steel, copper, aluminum and brass.

37. The shaft member of claim 35, wherein the proximal end of at least one of the inner tube and the outer tube is removably coupled to the handle assembly.

38. The shaft member of claim 35, wherein the distal end of at least one of the inner tube and the outer tube is removably coupled to the tissue engaging means.

39. The shaft member of claim 35, wherein the inner tube and the outer tube are formed as a co-extrusion.

40. A surgical device comprising:
a tissue engaging means;
a handle assembly;
an actuating means connecting the handle assembly and the tissue engaging means for actuating the tissue engaging means;
a shaft member made of a malleable material and having a proximal end, a distal end and a longitudinal axis, the proximal end of the shaft member coupled to the handle assembly, the distal end of the shaft member coupled to the tissue engaging means, the actuating means extending axially through the shaft member, the shaft member configured to be kink resistant, fatigue resistant, and to bend about a bending radius in response to a bending moment applied to the shaft member; and
an outer tube having a proximal end, a distal end and a longitudinal axis, the outer tube coaxially aligned with the shaft member, the shaft member disposed within the outer tube.

41. The surgical device of claim 40, wherein the outer tube is made of a material selected from the group consisting of aluminum, brass, copper and plastic, and wherein the shaft member is made of a material selected from the group consisting of stainless steel, copper and brass.

42. The surgical device of claim 40, wherein the proximal end of at least one of the shaft member and the outer tube is removably coupled to the handle assembly.

43. The surgical device of claim 40, wherein the distal end of at least one of the shaft member and the outer tube is removably coupled to the tissue engaging means.

44. The surgical device of claim 40, wherein the shaft member and the outer tube are formed as a co-extrusion.

45. A shaft member for a surgical device having a tissue engaging means and a handle assembly, and an actuating means connecting the handle assembly and the tissue engaging means for actuating the tissue engaging means, the shaft member comprising:
an elongate tubular member having a proximal end, a distal end, and an inner matrix, the proximal end of the tube coupled to the handle assembly, the distal end of the tube coupled to the tissue engaging means, the inner matrix defining a plurality of longitudinally extending lumens, at least one lumen extending from the proximal end to the distal end and configured to receive the actuating means, at least one additional lumen configured to receive an elongate malleable support member.

46. The shaft member of claim 45, wherein the elongate tube is made of a resilient material.

47. The shaft member of claim 46, wherein the plurality of lumens is five lumens and wherein a support member is disposed within four of the five lumens.

48. The shaft member of claim 45, wherein the support member is a metal cable.

49. A surgical device comprising:
a tissue engaging means;
a handle assembly;
an actuating means connecting the handle assembly and the tissue engaging means for actuating the tissue engaging means;
a shaft member having a proximal end, a distal end, and an inner matrix, the proximal end of the tube coupled to the handle assembly, the distal end of the tube coupled to the tissue engaging means, the inner matrix defining a plurality of longitudinally extending lumens, at least one lumen extending from the proximal end to the distal end and configured to receive the actuating means, at least one additional lumen configured to receive an elongate malleable support member.

50. The surgical device of claim 49, wherein the shaft member is made of a resilient material.

51. The surgical device of claim 50, wherein the plurality of lumens is five lumens and wherein a support member is disposed within four of the five lumens.

52. The surgical device of claim 49, wherein the support member is a metal cable.
